(19) United States
(12) Patent Application Publication
(10) Pub. No.: US 2004/0186489 A1
(43) Pub. Date: Sep. 23, 2004

(54) Anastomosis device

(76) Inventor: Hoon Bum Lee, Seoul (KR)

Correspondence Address: LADAS & PARRY, 5670 WILSHIRE BOULEVARD, SUITE 2100, LOS ANGELES, CA, 90036-5679, US

(21) Appl. No.: 10/478,765

(22) PCT Filed: May 31, 2002
(86) PCT No.: PCT/KR02/01039

(30) Foreign Application Priority Data
May 31, 2001 (KR) 2001-0030280

(51) Int. Cl. A61F 2/06

(52) U.S. Cl. 606/153

(57) ABSTRACT

The anastomosis device according to the present invention can be used to the end-to-end anastomosis of tubular organs as well as blood vessels, in particular, the blood vessels with different diameters which cannot be anastomosed with existing devices. The anastomosis device can be inserted into the operative site just through a small incision, which allows the endoscopic operation, whereby the operation time is remarkably lessened and the patient's pain is diminished. Further, the healing time is shortened, and the intentional heart attack may not be required for the cardiac operation. Moreover, without incising or excising the almost occluded or clogged region of a blood vessel, it is possible to connect only its normal regions to each other.

### TECHNICAL FIELD

The present invention relates to small devices for anastomosis of tubular organs such as blood vessels or fistulas, and more particularly anastomosis devices capable of easily anastomosing blood vessels of even different diameters or thickness as well as the same diameter and, in another embodiment, removing an occluded or clogged region of a vessel as a donut form and instead anastomosing normal regions thereof.

### BACKGROUND ART

The lack of exercise and occidentalization of eating habits are factors of increasing the incidence rate of vascular diseases from year to year. In particular, most of vascular diseases are cardiac infarction and angina pectoris caused by the ischemia that a blood vessels is clogged feeding nutriment and oxygen to the heart.

In order to treat the clogged (occluded) vessel or almost clogged vessel, a pharmaco-therapy using muscle relaxants or anti-calcium drugs for expansion of vessel muscle and an operative-therapy have been practiced. The pharmaco-therapy is useful for treatment at the initial symptom but is not so when the clogged step was progressed to the fair extent; therefore, the pharmaco-therapy is not substantial treatment and the operative-therapy has been generally practiced being the direct treatment of clogged vessel.

The operative-therapy is categorized into an internal operation and a surgical operation. The internal operation is to insert a stent into the clogged region (atresia region) through a blood vessel such as the femoral vessel and then expand the stent. The method of using an expansible stent and apparatuses therefor are disclosed in U.S. Pat. No. 3,416,531. However, when a certain time elapses after the internal operation according to such method, lesion tissues protrude between nets of the stent and new granular tissues grow on the inner surface of stent, whereby the atresia phenomenon reoccurs.

Accordingly, as a further essential solution to this problem, the surgical operation has been carried out opening the heart and connecting the atresia vessel by a bypass with a vessel harvested from other organ of a patient (mainly, leg vein). In this surgical operation, one end of the vein is connected to IMA (Internal Mammary Artery: the artery feeding nutriment and oxygen to the organ and muscle of the chest and abdomen) by the end-to-end or end-to-side way, and the other end is connected to the vessel beyond the atresia region by the end-to-side or side-to-side way.

Meanwhile, a blood vessel comprises the intima, the media and the adventitia, and, in anastomosis of two vessels, the intima should be connected to the intima with these vessels everted. For anastomosis of severed vessels, and reconstruction by the skin flap transplantation, as well as treatment of cardiac diseases like the above, the specialist in the microsurgery secures a magnified operative visual-field with a microscope or a powerful magnifier and then sutures vessels or tissues one by one with a suture (stitching fiber). The suture can be carried out only by the microsurgical specialist and thus is very time-consuming and heavy work. In particular, it is very difficult to suture one by one vessels being in the portion like the heart which pulsates periodically. Accordingly, it is necessary to stop the pulsation of heart for a long time (at least more than 3 hours) by causing the heart attack intentionally in operation.

In order to avoid the procedure of suturing vessels one by one with a stitching fiber by hand, a plurality of vessel anastomosis devices were made. Examples for the end-to-end anastomosis device of vessel are disclosed in U.S. Pat. No. 3,774,615, U.S. Pat. No. 4,214,586 and U.S. Pat. No. 4,917,087 and a commercial example is the microvascular anastomotic coupler from 3M corporation. These devices generally comprise rings of fixed frame and fixing pins and can be used only to two vessels having the same diameter. In other words, these devices cannot be used in anastomosis of vessels having different diameters or thickness.

FIG. 1 shows the blood flows in vessels of different diameters anastomosed by two ways. For connecting a vessel 100 with a relatively small diameter and a vessel 200 with a large diameter, as mentioned above, the intima of small-diameter vessel 100 must be contacted to the intima of large-diameter vessel 200 and also the connected portion must be continuous as in FIG. 1(a) in view of the hemodynamics. If the anastomosis is in a non-continuous connect as in FIG. 1(b), the whirling occurs thereby causing thrombus which is accumulated on the connected portion and resultantly occluding the vessel. The thrombus may cause the occlusion of ophthalmic artery or vein by clogging micro-vessels (representatively, brain vasculature), thereby causing new diseases such as amblyopia, blindness, palsy and the like.

End-to-end type vascular anastomosis can be classified into the anastomosis of two vessels with the same diameter and the anastomosis of two vessels with different diameters, and most of vascular anastomosis pertains to the latter. Therefore, although a plurality of devices as above were developed, most of vascular anastomosis operations are now conducted by micro surgical specialists who suture directly vessels with a stitching fiber.

So far, prior arts and their problems regarding only the vascular anastomosis have been described but these problems or almost similar problems also occur in the case of anastomosis of tubular organs.

### SUMMARY OF INVENTION

The objects of the present invention are to solve the problems described above for once and all.

That is, an object of the present invention is to provide an anastomosis device capable of anastomosing blood vessels or tubular organs having different diameters as well as the same diameter by the end-to-end way.

A further object of the present invention is to provide an anastomosis device capable of carrying out the endoscopic operation through a small incision of about 0.5 ˜1.0 cm on the skin, without opening fully the operative site for securing the sufficient operation viability.

Another object of the present invention is to provide an anastomosis device capable of removing the occluded or almost clogged region of vessels or tubular organs and instead anastomosing the normal regions thereof easily.

In order to accomplish these objects, the anastomosis device of the present invention by the end-to-end way, comprises a pair of semi-cylindrical members capable of extending to a certain length in applying a pulling force thereto and being disassembled/assembled,

wherein each semi-cylinder member comprises a plurality of assemblage parts, the top and/or bottom assemblage part having on its vertical plane a vertical coupler of being joined to the assemblage part of the corresponding semi-cylindrical member, and,

each assemblage part has extensible connecting parts of connecting itself to adjoining assemblage parts but extending in applying a pulling force thereto, and has on its lateral plane lateral couplers of being joined to the assemblage part of the corresponding anastomosis device.

The anastomosis device of the present invention is converted to a ring when a pair of semi-cylindrical members is coupled to each other. The coupled semi-cylindrical members (i.e., one ring) can also be joined to the other coupled semi-cylindrical members (the other ring) at the lateral side.

The term “lateral” used in the present specification means, as seen in FIG. 3A, a direction toward the circular cross-sectional plane of the coupled semi-cylindrical members (the xz plane of space coordinates), and the term “vertical” means a direction toward its vertical plane (the yz plane of space coordinates).

The vertical coupler used to couple a pair of semi-cylinder members may have the same configuration with the lateral coupler used to join two of the coupled anastomosis devices, or may not do so. Preferably, these couplers have the configuration being able to be joined by applying a small force at one time without any complicate handling in consideration that the anastomosis is conducted in the delicate operation such as a vascular operation. Accordingly, the couplers comprise preferably a pair of male and female parts, wherein the male part, for example, protrudes as the anchor form (both arms of the anchor show off the spring effect) and the female part caves for insertion of the male part. In the case that the corresponding portion to be inserted by the male part is made of a somewhat soft material, that portion needs not cave because the male part can be easily inserted into the soft material.

In another embodiment, the tops of both semi-cylindrical members are connected by a connecting member and each bottom assemblage part of them has a vertical coupler being able to be disassembled and assembled. Such configuration is similar to that of a handcuff. Therefore, both semi-cylinder members can pivot on the axis of the upper connecting member, whereby both semi-cylinder members can be easily placed on the vessel and then fastened.

The extensible connecting part which acts as connecting neighbor assemblage parts to each other can extend by a small pulling force. Resultantly, the diameter of ring formed by the coupled semi-cylindrical members can become larger by a small pulling force. The preferred configuration of assemblage parts and extensible connecting parts, being able to extend by a small pulling force as described above, is that the assemblage part is hollow and has apertures for movement of the extensible connecting parts on both ends toward adjoining assemblage parts, and the extensible connecting part comprises a body of moving through the aperture and lugs having the bigger dimension than the diameter of the aperture. As such, when any pulling force is not applied, two neighbor assemblage parts adjoin to each other at their ends and connected by the extensible connecting part passing through their apertures. To the contrary, when the pulling force is applied, the ends of neighbor assemblages become more distant. The maximum distance of separation is defined with the distance that the lug can reach the aperture of assemblage.

Since the anastomosis device of the present invention is a product to be inserted in the human body, it must be made of biocompatible materials. For example, the assemblage parts and connecting parts may be made of silicone, titanium, or PGLA (blend of Polyglycolic acid and Polylactic acid) being decomposed after lapse of a certain time in vivo, and the couplers may be made of titanium harmless to the human body.

The anastomosis device of the present invention may be used to the end-to-end anastomosis of tubular organs as well as blood vessels. These tubular organs have a tubular structure, for example, fallopian tube, bile duct, bowels, parotid duck, urethra, ureter, spermatic cord, ductus pancreaticus, ductus hepaticus communis, fallopian tube, lymphatic duct, etc.

As shown below, the description refers to the drawing in order to describe the present invention more in detail, thereby, the scope of the invention is however not to be interpreted as a limitation of the invention.

### DETAILED DESCRIPTION OF PREFERRED EMBODIMENTS

FIGS. 2 to 4 show the perspective and cross-sectional views of a pair of anastomosis devices and their coupled and extended shapes. Referring to FIG. 2A, a pair of anastomosis devices can be disassembled and consist of left/right anastomosis devices 302, 304 with the same configuration. The left/right anastomosis devices 302, 304 comprise a plurality of assemblage parts 310. Each of the assemblage parts 310 has a lateral coupler 400 on its lateral plane wherein the lateral coupler 400 consists of a male part 402 and a female part 404. In a top assemblage part 312 of the left anastomosis device 302, a male coupler 402 to join to the corresponding assemblage part of the right anastomosis device 304 is installed on its vertical plane. To the contrary, in a bottom assemblage part 314, a female coupler 404 to join to the corresponding assemblage part of the right anastomosis device 304 is installed on its vertical plane. The right anastomosis device 304 is the same. The male coupler 402 with the form of anchor has both arms to easily join to the female coupler 404 by a spring effect.

Referring to FIG. 2B, another anastomosis device 300′ is illustrated that its top is connected but its bottom is separated. In another embodiment, in order to easily bring the anastomosis device 300′ to the operative site such as a blood vessel or tubular organ, a protruding part 340 to be hung to other auxiliary device (not shown) is installed on a bottom assemblage part 314. The configuration of the protruding part 340 is not limited to one depicted in FIG. 2B.

FIGS. 3A and 3B show the perspective view and horizontal cross-sectional view of the coupled anastomosis devices 300 in the state that a pair of anastomosis devices are coupled to each other. The coupled anastomosis devices 300 form a ring. Each assemblage part 310 is hollow and has apertures 320 on its both ends. Each assemblage part 310 is connected to neighbor assemblage parts by extensible connecting parts 500, and one extensible connecting part 500 is connected to two assemblage parts 310 through the apertures 320. The extensible connecting part 500 consists a connecting body 510 with a certain length and two lugs at its both ends, wherein the dimension of the lug is larger than the diameter of the aperture 320 of the assemblage part 310.

FIGS. 4A and 4B show the perspective view and cross-sectional view of the coupled anastomosis devices 300 in the state that the coupled anastomosis devices 300 are expanded by an applied pulling force. Assemblage parts 310 are separated to each other, and the maximum distance of separation corresponds to the length of a body 510. At the maximum expansion, a lug 520 of an extensible connecting part 500 reaches an aperture 320 of the assemblage part 310.

In below, the procedure of employing the device of the present invention to the anastomosis of blood vessels is illustrated referring to FIGS. 5A to 5D. Herein, the anastomosis device 300 of the present invention is employed to anastomosis of the vessels with different diameters.

Referring to FIG. 5A, a pair of coupled anastomosis devices 300 are fastened to a relatively small-diameter vessel 100 and a large-diameter vessel 200. Preferably, the inner diameter of the anastomosis device 300 is smaller than the diameter of the small-diameter vessel 100. Accordingly, the vessels 100, 200 fastened by the anastomosis device 300 become narrower in their fastened portion.

For anastomosis of vessels, as mentioned above, the intima of the vessel must be connected to the intima of the corresponding vessel with each end of vessels everted. As such, it is necessary to evert the vessels to expose their intimas, as seen in FIG 5B. Since the inner diameter of the anastomosis device 300 is smaller than the diameters of vessels 100, 200, it is easy to evert the ends thereof. As the vessels 100, 200 are everted, the tip of a lateral male coupler 410 on an assemblage part pierces the vessels 100, 200.

In the next step, the coupled devices 300 wrapped with vessels 100, 200 are joined as seen in FIG. 5C. Although it cannot be seen in the drawing, the lateral male coupler (not shown) of the assemblage parts penetrates the vessels 100, 200 to be inserted into the corresponding assemblage part. In the case that the assemblage part are made of a somewhat soft silicone, the male coupler can be inserted to the corresponding assemblage part on which any female coupler is not formed.

As the blood is supplied after joint of two coupled anastomosis devices 300, the diameters of these coupled devices 300 become larger by the pressure of blood flow. At this time, the coupled anastomosis devices 300 fastened on the vessel 100 are expanded to the same extent that the coupled anastomosis device 360 fastened on the vessel 200 are expanded.

In order to diminish the loss of blood by hemorrhage at the anastomosis of vessels and facilitate the operation, it is preferable to position a blocking device of cutting off the blood flow at the rear of the portion fastened by the device 300 during the anastomosis to block the flow of blood. As an example of such device, FIG. 6 shows tongs 600 holding the vessels. Although it is not seen in the drawing, the tongs 600 also holds the large-diameter vessel 200. The tongs 600 will be removed after completion of the anastomosis procedure, i.e., after the step of FIG. 5C.

FIGS. 7A to 7D show a procedure of removing the almost clogged region of a blood vessel by employing the anastomosis device 300 of the present invention. The almost clogged region of blood vessel in these drawings is entirely removed; however, it is also possible to anastomose the normal regions without removal of the almost clogged region.

Referring to FIG. 7A, when the atresia occurs on the specific blood vessel, it is necessary to remove a clogged region 112 of a blood vessel 110 and then connect normal regions 114, 116 to each other. In this case, two coupled anastomosis devices 300 of the present invention are fastened on the normal regions 114, 116 respectively, and then the normal regions 114, 116 and the clogged region 112 are sufficiently expanded by using a catheter (not shown). At this time, the inner diameter of the coupled anastomosis devices 300 should be smaller than the diameter of the blood vessel prior to expansion. Accordingly, as seen in FIG. 7B, the inner pressure of a blood flow affects the regions 112, 114, 116 narrowed by the coupled anastomosis devices 300, whereby these regions are expanded. In particular, the clogged region 112 is further expanded because of its smaller inner diameter, in comparison with the normal regions fastened by the anastomosis device 300. At this time, both coupled anastomosis devices 300 are joined to each other, as in FIG. 7C. As these coupled anastomosis devices 300 are joined, the clogged region 112 protrudes. Referring to FIG. 7D, after joint of the anastomosis devices 300, these anastomosis devices 300 are expanded by the pressure of the blood flow, whereby the normal regions 114, 116 are connected to each other. The clogged region 112 may be removed or may be left if necessary.

The present invention being thus described, it will be obvious that the same may be varied in many ways. Such variations are not to be regarded as a departure from the spirit and scope of the invention and all such modifications would be obvious to one skilled in the art.

### BRIEF DESCRIPTION OF THE DRAWINGS

FIG. 1 is a cross-sectional view of showing the blood flow of the vessels with different diameters, which are anastomosed in different anastomosis ways.

FIG. 2A is a perspective view of a pair of anastomosis devices according to the present invention.

FIG. 2B is a perspective view of another anastomosis devices according to the present invention, in which the top of anastomosis devices is connected by a connecting member.

FIG. 3A is a perspective view of the coupled anastomosis devices of FIG. 2A.

FIG. 3B is a cross-sectional view of FIG. 3A.

FIG. 4A is a perspective view of the anastomosis device of FIG. 3A expanded by a pulling force.

FIG. 4B is a cross-sectional view of FIG. 4A.

FIGS. 5A to 5D are procedure views of anastomosing blood vessels with different diameters employing the anastomosis device of FIG. 2A.

FIG. 6 is a step view in the case of using tongs, which corresponds to the step view of FIG. 5A.

FIGS. 7A to 7D are procedure views of removing an almost clogged region of a certain blood vessel as a donut form and instead connecting normal regions to each other employing the anastomosis device of FIG. 2A.

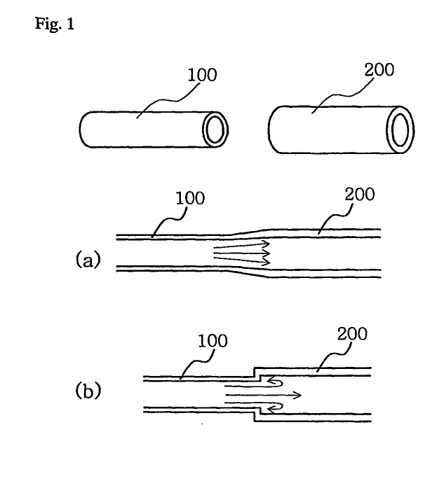

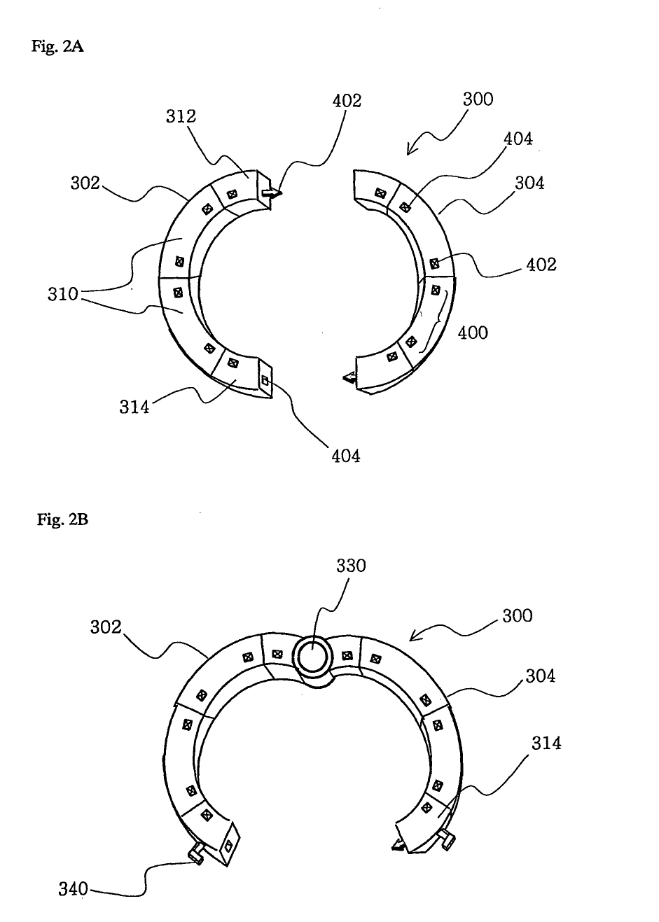

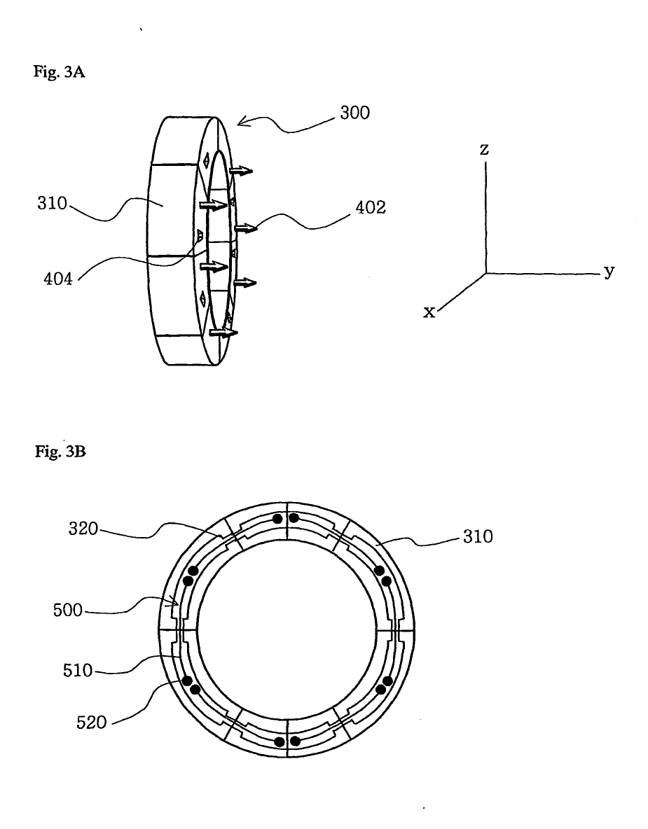

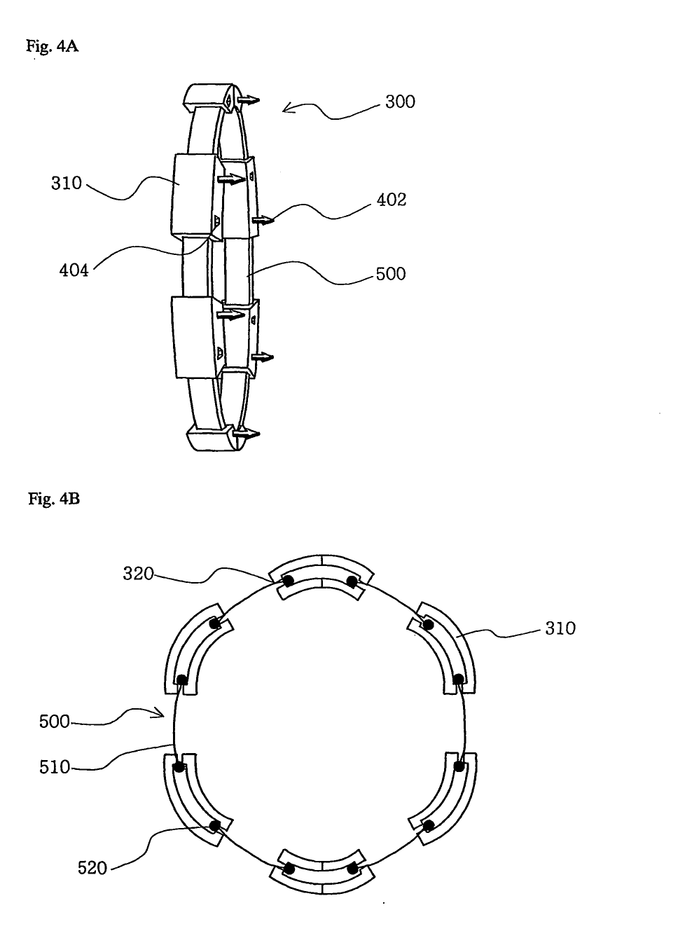

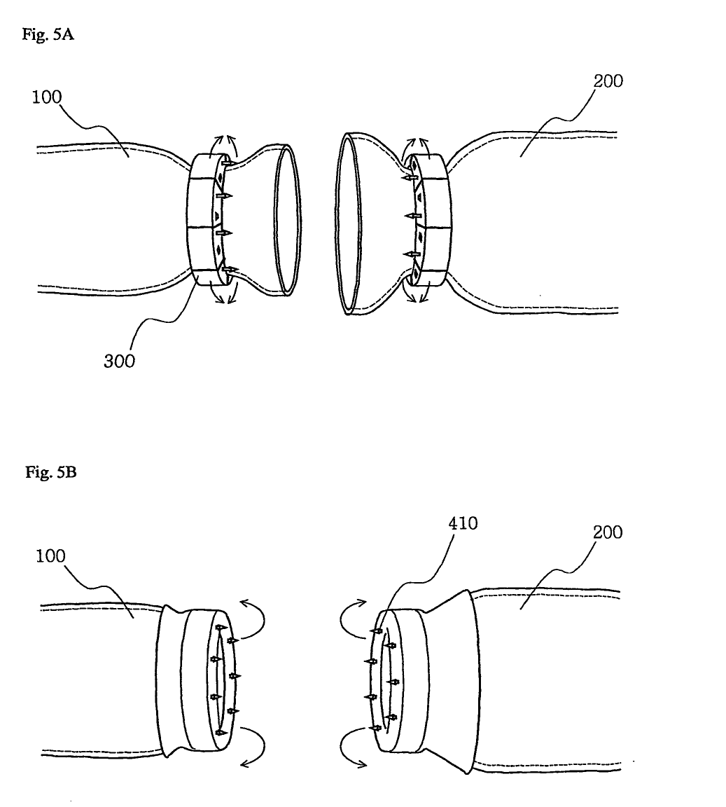

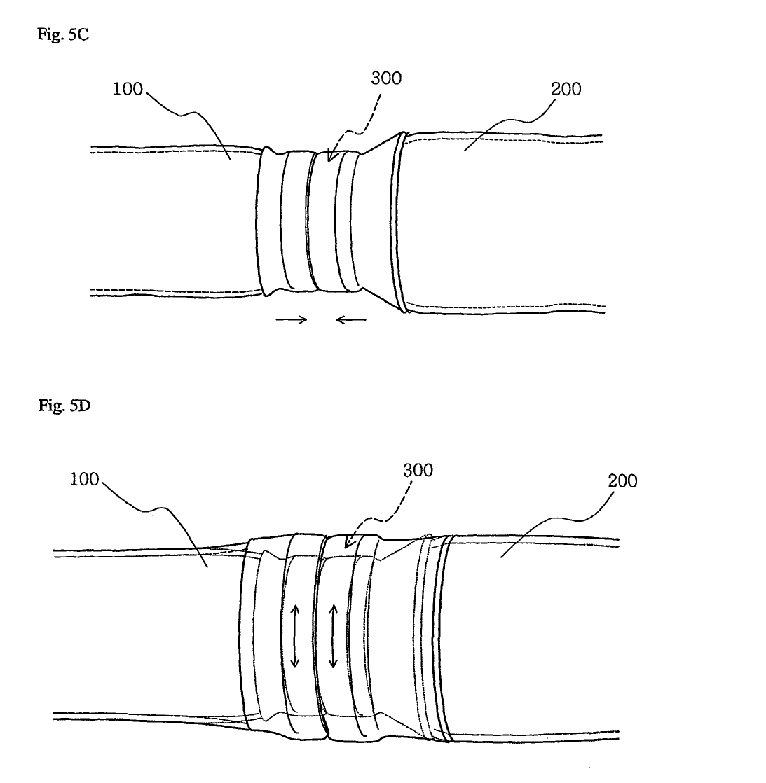

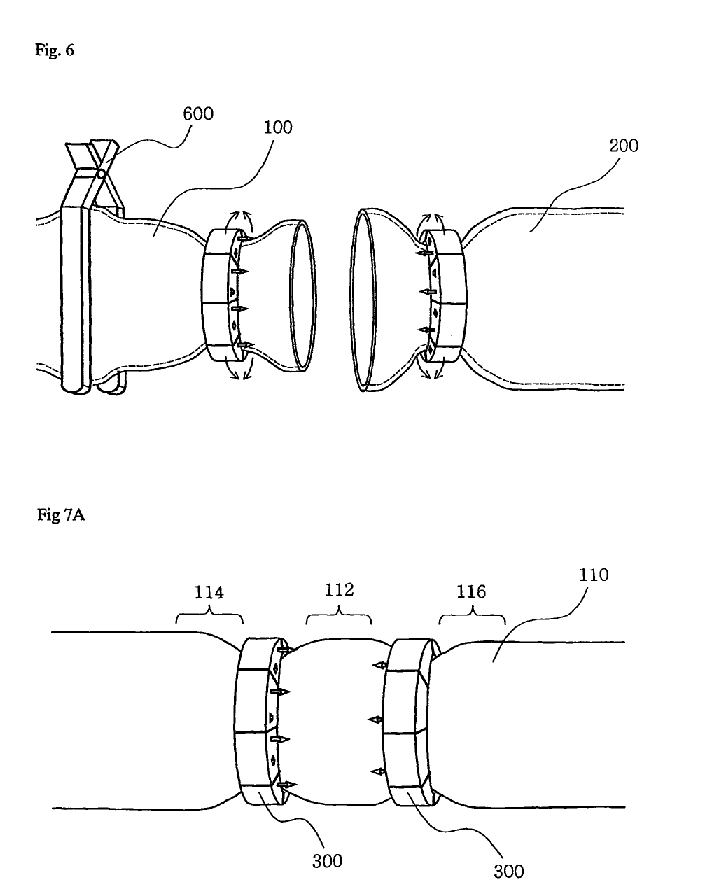

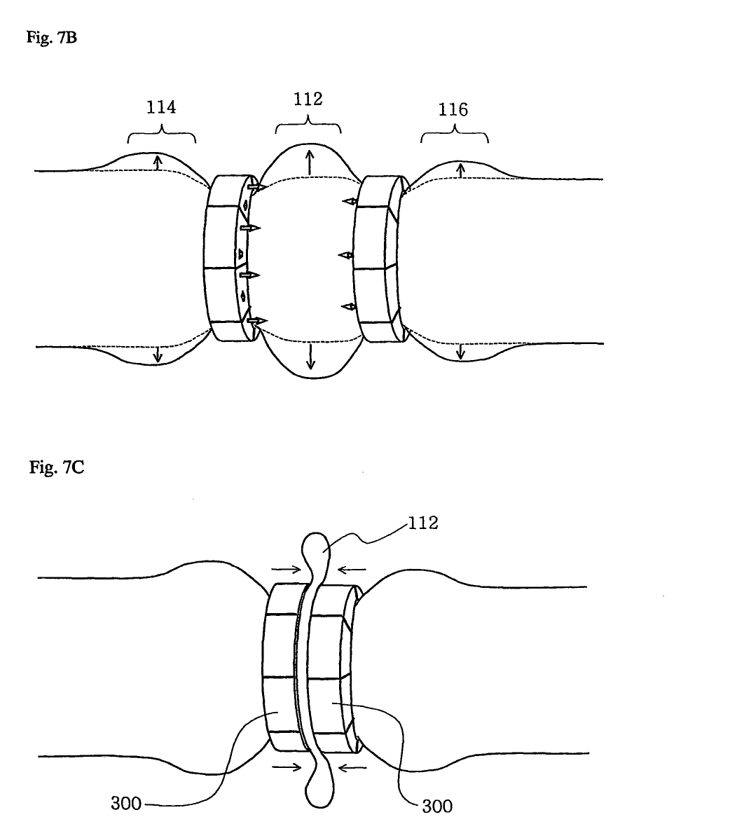

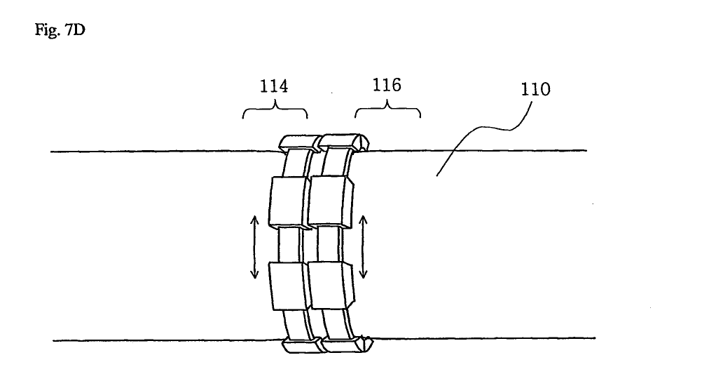

### INDUSTRIAL APPLICABILITY

The anastomosis device according to the present invention can be used to the end-to-end anastomosis of tubular organs as well as blood vessels, in particular, the blood vessels with different diameters which cannot be anastomosed with existing devices. The anastomosis device can be inserted into the operative site just through a small incision, which allows the endoscopic operation, whereby the operation time is remarkably lessened and the patient's pain is diminished. Further, the healing time is shortened, and the intentional heart attack may not be required for the cardiac operation. Moreover, without incising or excising the almost occluded or clogged region of a blood vessel, it is possible to connect only its normal regions to each other.

What is claimed is:

1. Anastomosis device comprising,
a pair of semi-cylindrical members capable of extending to a certain length in applying a pulling force thereto and being disassembled/assembled,
wherein each semi-cylindrical member comprises a plurality of assemblage parts, the top and/or bottom assemblage part having on its vertical plane a vertical coupler of being joined to the assemblage part of the corresponding semi-cylindrical member, and,
each assemblage part has extensible connecting parts of connecting itself to adjoining assemblage parts but extending in applying a pulling force thereto, and has on its lateral plane lateral couplers of being joined to the assemblage part of the corresponding anastomosis device.

2. The anastomosis devices according to claim 1, wherein the vertical coupler and the lateral coupler have the same or different configurations and comprise a pair of male and female parts, the male part protruding as the anchor form and the female part caving for insertion of the male part.

3. The anastomosis devices according to claim 1 or 2, wherein the tops of both semi-cylindrical members are connected by a connecting member and each bottom assemblage part thereof has a vertical coupler being able to be disassembled and assembled.

4. The anastomosis devices according to claim 1 or 2, wherein the assemblage part is hollow and has apertures for movement of the extensible connecting parts on both ends toward adjoining assemblage parts, and the extensible connecting part comprises a body of moving through the aperture and lugs having the bigger dimension than the diameter of the aperture.

5. The anastomosis devices according to claim 1 or 2, wherein the devices is employed for anastomosis of the blood vessels.
